(19) United States
(12) Patent Application Publication
(10) Pub. No.: US 2010/0198176 A1
(43) Pub. Date: Aug. 5, 2010

(54) ADHESIVE COMPONENT FOR OSTOMY DEVICES

(76) Inventors: Eben Stroebech, Hoersholm (DK); Anders Bach, Kobenhavn (DK); Mads Lykke, Broenshoej (DK); Astrid Toftkaer, Soeborg (DK); Hasse Buus, Humlebaek (DK); Torn Kongebo, Humlebaek (DK)

Correspondence Address: JACOBSON HOLMAN PLLC, 400 SEVENTH STREET N.W., SUITE 600, WASHINGTON, DC, 20004, US

(21) Appl. No.: 12/452,508

(22) PCT Filed: Jun. 17, 2008
(86) PCT No.: PCT/DK2008/050144

§ 371 Date: Apr. 1, 2010

(30) Foreign Application Priority Data
Jul. 6, 2007 (DK) PA2007 01003
Apr. 17, 2008 (DK) PA 2008 00561

(51) Int. Cl. A61L 24/06; A61L 24/04; A61F 5/448

(52) U.S. Cl. 604/344; 523/105

(57) ABSTRACT

A component for sealing a body opening and for use in combination with a body waste collecting device, said component comprises a soft adhesive composition wherein the soft adhesive composition comprises a liquid impermeable, moisture permeable adhesive composition.

### FIELD OF THE INVENTION

The present invention relates to a collecting device for attachment to human skin and for collecting bodily waste e.g. an ostomy device.

### BACKGROUND

Collecting devices for collecting bodily waste, ostomy appliances, wound or fistulae drainage bandages or devices for collecting urine are usually in the form of a receptacle, e.g. a bag, pouch or tube for receiving the waste, connected to an adhesive wafer that can be attached to the skin of the patient. The wafer is typically in the form of a backing layer coated on the skin-facing surface with an adhesive layer and the wafer may further be provided with an aperture for accommodating the body opening. The size and shape of said aperture can often be adapted individually to fit the anatomy of the patient.

One of the crucial parts of such devices is the adhesive wafer. The wafer should be able to fit leak proof around the body opening, have good adherence to the skin without unintended detachment from the skin, but at the same time be easy to remove again without damaging the skin. Furthermore, the wafer should be able to follow the movements of the body and be comfortable to wear.

When designing a skin adhesive one of the major issues is to keep the skin relatively dry underneath the adhesive to prevent maceration. Maceration occurs when skin is unable to get rid of moisture from transpiration and outlet from a body opening. This may result in degradation of the skins barrier function as well as bad adhesion of the device to skin.

Usually, skin adhesive keeps the skin dry by absorbing moisture. This allows moisture to transport through the adhesive from the skin side to the opposite side, where it is allowed to evaporate.

Adhesives for fastening a bodily waste collecting pouch or tube are usually covered by a moisture impermeable layer that does not allow moisture from the skin to permeate through the adhesive and out into the surroundings.

Therefore, adhesives for fastening a collecting pouch or tube are made moisture absorbent. Absorbing particles or hydrocolloids (HC) are mixed into an adhesive matrix in order to absorb moisture from the skin and thereby the skin is kept relatively dry. This technique is well known in the art and forms the basis for most commercially available ostomy adhesives see, e.g. U.S. Pat. No. 4,192,785.

The major problem in using an absorbing adhesive is the changes in the bonding properties when the adhesive absorbs moisture. An absorbing adhesive normally starts out being a well-bonded adhesive and usually ends up being a weak-bonded adhesive after absorbing moisture. This effect is particularly a problem for hydrocolloid-based adhesives. As the adhesive absorbs moisture from the skin, the hydrocolloids swell and take up an increasing amount of space in the skin-bonding zone, thereby reducing the skin bonding effect of the adhesive.

The weak-bonded adhesive will usually still keep the collecting device attached to the skin, however the risk of leakage of bodily waste along the skin surface will increase drastically.

Hence, there is a need to find a solution to avoid leakage of bodily waste occurred because of weak-bonded adhesive and still maintain the ability to use conventional waste collecting devices to adhere to the skin.

It has now surprisingly been found that the component according to the invention provides a sealing element that can be used in combination with conventional waste collecting devices.

### SUMMARY OF THE INVENTION

The present invention relates to a component for sealing a body opening and for use in combination with a body waste collecting device, said component comprises a soft adhesive composition wherein the soft adhesive composition comprises a liquid impermeable, moisture permeable adhesive composition.

### BRIEF DESCRIPTION OF THE DRAWING

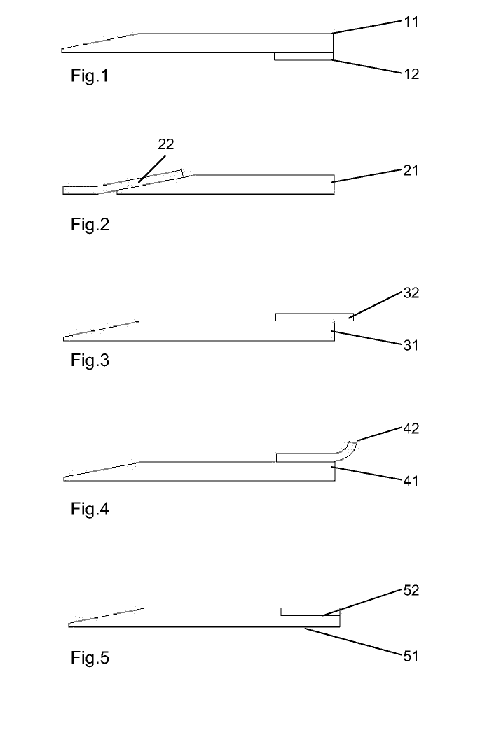

The invention is disclosed more in detail with reference to the drawings in which FIG. 1-5 show different embodiments of the invention.

### DETAILED DESCRIPTION OF THE PRESENT INVENTION

The aim of the invention is to provide a sealing component for use in combination with a body waste collecting device. The sealing component is to be used as a supplement to the collecting device.

By body waste collecting device is meant a device being able to collect and hold the output in a collecting item for a predefined time. The holding in place of the device may be obtained by a skin adhesive and the collection may be obtained by a bag.

In an embodiment of the invention a component for sealing a body opening and for use in combination with a body waste collecting device, comprises a soft adhesive composition wherein the soft adhesive composition comprises a liquid impermeable, moisture permeable adhesive composition.

The component comprising the soft adhesive composition is used for security and protection for the user in order to avoid leakages and to obtain longer wear time.

Standard pressure sensitive adhesives used today are more or less plastic, which means that the adhesive will get a good grip by flowing into cavities of the skin mantles. This results in a large adhesion area and thus a good skin adhesion enhanced by the mechanical anchoring.

A standard and conventional hydrocolloid pressure sensitive adhesive is a skin friendly adhesive that is capable of adhering to the skin, handling perspiration by absorbing the moist and being removable from the skin without essential damage.

A typical pressure sensitive adhesive is based on a polyisobutylene, PIB (adhering substance) and a carboxy methyl cellulose, CMC (absorbing media).

When using conventional hydrocolloid containing adhesives, the adhesive wafer tends to be relatively stiff caused by the choice of adhesive materials as well as the high filling rate of hydrocolloids (typical 30-60% w/w). Incorporation of hydrocolloids ensures that the perspiration of moisture from the skin is absorbed in a way that the skin will not macerate due to constant water exposure. As a side effect the hydrocolloids tend to swell from exposure of the water containing output. In general the tack of a hydrocolloid adhesive tends to decrease due to the absorption of the moisture perspiration from the skin into the adhesive.

Even with the decreased tack, the adhesive will usually keep the collecting device attached to the skin where the device is applied. Unfortunately, however, the decreased tack may cause a leakage of body fluids from the body opening along the skin surface.

The component according to the invention compensates for this leakage failure using the mechanical and adhesive properties of a liquid impermeable, moisture permeable adhesive, thus achieving a solution to the problem of conventional waste collecting devices.

A liquid impermeable, moisture permeable adhesive does not absorb the moisture but rather permeates the water away from the skin surface.

By permeable adhesive is meant an adhesive where the moisture vapour transmission rate, MVTR is higher than 100 g/m²/24 h as measured using the method disclosed herein.

The adhesive used in the component of the present invention has a high moisture vapour transmission rate, preferably a MVTR over 100 g/m²/24 hrs, which makes it breathable and very skin friendly. The high moisture transmission of the adhesive is a particular advantage where a medical device has to be worn on the skin for a long time, e.g. days.

Another important property of the component of the invention is that the adherence force of the soft adhesive used does not change with time, or changes only to a small extent with time, during wear time of the device.

The component comprising a liquid impermeable, moisture permeable adhesive composition gives a soft, tight and pliable adhesive in the perianal, peristomal or fistal area with optimal protection. The soft adhesive offers a virtually non-erosive and non-swellable solution yet a skin friendly breathable, liquid impermeable, moisture permeable adhesive.

The fixation of the component will add skin friendliness and extreme flexibility giving the user of the collecting device an optimal security and non-traumatic solution.

According to one embodiment of the invention, the soft adhesive composition comprises the liquid impermeable, moisture permeable adhesive composition, which comprises a permeable polymer selected from the group of but not limited to polypropyleneoxide, polyurethane, silicone, polyacrylate, ethylene vinyl acetate and mixtures thereof.

As used herein a permeable polymer means a polymer that absorbs less than 5% in wt, preferably less than 1%, at equilibrium and has a moisture vapour transmission rate greater than 100 g/m²/24 hrs, preferably greater than 200 g/m²/24 hrs.

According to an embodiment of the invention, the water vapour permeability of the soft adhesive composition comprising the liquid impermeable, moisture permeable adhesive composition is higher than 100 g/m²/24 h as defined herein.

As used herein liquid impermeable means that water is rejected from penetrating the substance by simple flow.

In one embodiment of the invention, the permeable polymer is crosslinked.

As used herein a crosslink means a small region in a macromolecule (polymer chain structure) from which more than 2 chains emanate. The linking may be covalent, physical or ionic.

In another embodiment of the invention, the liquid impermeable, moisture permeable adhesive composition comprises a block copolymer.

As used herein a block copolymer means a copolymer in which the repeating units in the main chain occur in blocks, e.g., -(a)m-(b)n-(a)p-(b)q-, where a and b represent the repeating units and m, n, p, q, are numbers.

In a preferred embodiment of the invention, the liquid impermeable, moisture permeable adhesive composition comprises polypropyleneoxide.

In a preferred embodiment of the invention, the liquid impermeable, moisture permeable adhesive composition comprises polyurethane.

In a preferred embodiment of the invention, the liquid impermeable, moisture permeable adhesive composition comprises ethylene vinyl acetate.

The adhesive composition comprising ethylene vinyl acetate may suitably be an adhesive known in the art such as the adhesive composition disclosed, for example in Danish patent application PA 2007 01003.

In a preferred embodiment of the invention, the liquid impermeable, moisture permeable adhesive composition comprises silicone.

In a preferred embodiment of the invention, the liquid impermeable, moisture permeable adhesive composition comprises polyacrylate.

It may be advantageous that the soft adhesive composition comprises absorbent particles. According to an embodiment of the invention, the soft adhesive composition comprises absorbent particles.

The particles may be absorbent articles such as salts, hydrocolloids, microcolloids and/or super absorbers in order for the composition to absorb moisture from skin.

Microcolloid particles are well-known in the art, e.g. from International Patent Application No. WO 02/066087, which discloses adhesive compositions comprising microcolloid particles. The microcolloid particles may have a particle size of less than 20 microns.

The soft adhesive composition may comprise 1-30% w/w of hydrocolloid (HC) or super absorbent particles (SAP) particles, more preferred 5-30% w/w particles.

In the conventional adhesive constructions a high amount of hydrocolloids are needed in order to obtain an absorption into the adhesive. The reason for this is that the polymer matrix is impermeable to moisture so the filler particles have to be packed very close or in contact to each other. The absorption into the adhesive will occur through direct water transfer between two absorbing particles or by absorption in one particle resulting in a volume increase that generates contact to the next particle.

The absorption according to this embodiment of the invention has a polymer matrix that is permeable to moisture in a way that moisture will permeate through the polymer matrix into the absorbing particles. In order to obtain the same absorption speed, less absorption particles are then needed.

Salt may be advantageous to use as absorber in the component of this invention. A salt like sodium chloride has an equilibrium vapour pressure of about 75% RH at skin temperature and will absorb water from skin and output because of the difference in vapour pressure.

In an embodiment of the invention the soft adhesive composition comprises particles of mineral salt. The salt may be present in an amount of 1-50% w/w, more preferred in an amount of 5-25%.

The salt can be an inorganic salt or an organic salt.

According to one embodiment of the invention, the soft adhesive composition comprises water soluble inorganic salt from the group of but not limited to NaCl, CaCl₂, K₂SO₄, NaHCO₃, Na₂CO₃, KCl, NaBr, NaI, KI, NH₄Cl, AlCl₃ and mixtures thereof, preferably NaCl.

According to another embodiment of the invention, the soft adhesive composition comprises water soluble organic salt from the group of but not limited to CH₃COONa, CH₃COOK, HCOONa, HCOOK and mixtures thereof.

As used herein a paste is a moldable or flowable adhesive that is able to adhere to the skin, flow or being permanently pressed into irregularities of the skin and thus protecting the skin from the output. A paste is usually used as an accessory to stoma care products for sealing around the stoma.

Sealing an opening refers to protection of an opening from output for example use of a paste around a stoma at the rim of the perineal skin or the rim of a fistula.

The component for sealing the body opening can have elastic or plastic characteristics.

According to one embodiment of the invention, the collecting device comprises a collecting pouch and an adhesive wafer.

In an embodiment of the invention, the component is located on the skin-facing surface of the adhesive wafer.

By the skin-facing surface of the adhesive is meant the side adhering to the skin.

By located is meant where the component is placed on the collecting device and specifies the position of the component on the collecting device.

In another embodiment of the invention, the component is located on the pouch-facing surface of the adhesive wafer.

By the pouch-facing surface or non-skin-facing surface is meant the side of the adhesive or backing pointing away from the skin (non-bonding side).

According to another embodiment of the invention, the component is located at the inner rim of the adhesive wafer.

By the inner rim portion of the adhesive wafer is meant the portion in the perianal, peristomal or fistal area. This area of the adhesive is exposed to faeces as well as perspiration. The properties of the inner rim portion depend on wear time, output type and anatomy among others.

According to one embodiment of the invention the component is located at the outer rim of the adhesive wafer.

By the outer rim portion of the adhesive wafer is meant the portion essentially outside the welding zone or coupling. This portion is less exposed to faeces but has to handle perspiration as well as mechanical exposure due to movements and pull from the bag.

According to an embodiment of the invention the component is in the form of a ring.

By an adhesive ring is meant an adhesive, essentially surrounding the body opening that needs to be drained or a ring covering the outer or inner rim of the adhesive wafer.

According to an embodiment of the invention, a hole of the ring having a diameter that is less than the diameter of a hole in the adhesive wafer. By this embodiment the component will have a tighter fit around the body opening compared to the adhesive wafer. In one embodiment the sealing and the tighter fit of the component is obtained by turtle necking.

According to one embodiment of the invention, the component is part of a ring such as a half ring covering 180 degrees of a circle.

According to an embodiment of the invention, the component covers the entire skin-facing surface of the adhesive wafer.

Optionally, the component only covers a part of the adhesive wafer.

According to an embodiment of the invention, the component partly covers the adhesive wafer.

According to another embodiment of the invention, the component partly covers the skin-facing surface of the adhesive wafer.

According to another embodiment of the invention the adhesive wafer has a recess for adapting the component.

The collecting device could be pre-shaped with a recess that fits the component to be combined with the device. A recess could be a cut-out of part of the adhesive wafer.

By adapting is meant that the component is placed on the collecting device in a way that almost integrates the two separate parts.

The component according to the invention can be used in connection with adhesive wafer having a conventional hydrocolloid containing pressure sensitive adhesive usable for skin. However, the component is also usable in connection with other types of adhesives applied on the adhesive wafer.

According to an embodiment of the invention, the body opening is a stoma.

According to another embodiment of the invention, the body opening is a fistula.

According to another embodiment of the invention, the body opening is an anus.

By a body opening is meant a natural or artificial body opening such as an anus, a stoma, or a fistula.

According to an embodiment of the invention, the collecting device is an ostomy appliance.

According to another embodiment of the invention, the collecting device is a faecal collecting device.

According to another embodiment of the invention, the collecting device is a fistula collecting device.

### DESCRIPTION OF THE PREFERRED EMBODIMENTS

The invention is now explained more in detail with reference to the drawings showing preferred embodiments of the invention.

FIGS. 1-5 are all drawings of a side view of the left half of the adhesive wafer and the component according to the invention. All wafers are covered with a backing layer. The skin-facing surface is illustrated in the drawings as the downward side of the wafer and the backing layer is placed on top of the wafer.

FIG. 1 shows an adhesive wafer (11) and a component (12) where the component only covers part of the adhesive wafer and is placed on the surface of the skin-facing adhesive wafer. The component can optionally be covered by a second backing layer placed in between the non-skin-facing side of the component and the skin-facing side of the adhesive wafer.

FIG. 2 illustrates a side view of an adhesive wafer (21) partly covered with a component (22) in the outer rim portion of the wafer.

FIG. 3 illustrates a side view of an embodiment of the invention where the hole of the ring of the component (32) has a diameter that is less than the hole in the adhesive wafer (31).

FIG. 4 illustrates a side view of another embodiment of the invention where the hole of the ring of the component (42) has a diameter that is less than the hole in the adhesive wafer (41) and wherein the sealing is obtained by a turtleneck effect.

FIG. 5 illustrates a side view of an adhesive wafer (51) having a recess for adapting the component (52).

### EXPERIMENTAL PART

Methods

Determination of Water Absorption

In order to get better correlation between measured water absorption and actual performance in a humanlike environment, a modified version of the ISO 62 standard was used: Pieces of adhesive of 1×25×25 mm³ were fastened on a piece of glass using double sided adhesive and the constructs were immersed in saline water (0.9% NaCl in dematerialized water) at 32° C. After 24 hours the samples were removed and carefully dripped dry and weighed. The change in weight was recorded and reported as weight gain in percent of the original dry weight of the adhesive. In the following we will call this value w₂₄ₕ

Determination of Moisture Vapour Transmission Rate (MVTR)

MVTR was measured in grams per square meter (g/m²) over a 24 hours period using an inverted Paddington cup method (British Pharmacopoeia, 1993, Addendum 1996, page 1943. HMSO London): A container or cup being water and water vapour impermeable having an opening was used. 20 ml saline water (0.9% NaCl in demineralised water) was placed in the container and the opening was sealed with the test adhesive film. The container, with a duplicate, was placed into an electrically heated humidity cabinet and the container or cup was placed up side down in a way that the water was in contact with the adhesive. The cabinet was maintained at 37° C. and 15% relative humidity (RH). After about an hour, the containers were considered to be in equilibrium with the surroundings and it was weighed. 24 h after the first weighing, the containers were weighed again. The difference in weight was due to evaporation of vapour transmitted through the adhesive film. This difference was used to calculate Moisture vapour transmission rate or MVTR. MVTR was calculated as the weight loss after 24 h divided by the area of the opening in the cup (g/m²/24 h). If the adhesive film could not support the weight of the water, a supporting film with very high permeability was used as support.

1. A component for sealing a body opening and for use in combination with a body waste collecting device, said component comprises a soft adhesive composition, wherein the soft adhesive composition comprises a liquid impermeable, moisture permeable adhesive composition.

2. The component according to claim 1, wherein the soft adhesive composition comprises the liquid impermeable, moisture permeable adhesive composition, which comprises a permeable polymer selected from the group of polypropyleneoxide, polyurethane, silicone, polyacrylate, ethylene vinyl acetate and mixtures thereof.

3. The component according to claim 1, wherein the water vapour permeability of the soft adhesive composition comprising the liquid impermeable, moisture permeable adhesive composition is higher than 100 g/m²/24 h as defined herein.

4. The component according to claim 1, wherein the soft adhesive composition comprises absorbent particles.

5. The component according to claim 4, wherein the absorbent particles are selected from the group of salts, hydrocolloids, microcolloids, and super absorbers.

6. The component according to claim 1, wherein the collecting device comprises a collecting pouch and an adhesive wafer.

7. The component according to claim 6, wherein the component is located on the skin-facing surface of the adhesive wafer.

8. The component according to claim 6, wherein the component is located on the pouch-facing surface of the adhesive wafer.

9. The component according to claim 6, wherein the component is located at the outer rim of the adhesive wafer.

10. The component according to claim 6, wherein the component is located at the inner rim of the adhesive wafer.

11. The component according to claim 1, wherein the component is in the form of a ring.

12. The component according to claim 11, wherein a hole of the ring has a diameter being less than the diameter of a hole in the adhesive wafer.

13. The component according to claim 11, wherein the component is part of a ring.

14. The component according to claim 6, wherein the component partly covers the adhesive wafer.

15. The component according to claim 6, wherein the adhesive wafer has a recess for adapting the component.

16. The component according to claim 1, wherein the body opening is a stoma.

17. The component according to claim 1, wherein the body opening is a fistula.

18. The component according to claim 1, wherein the body opening is an anus.

19. The component according to claim 1, wherein the collecting device is an ostomy appliance.

20. The component according to claim 1, wherein the collecting device is a faecal collecting device.

21. The component according to claim 1, wherein the collecting device is a fistula collecting device.
